(19) United States
(12) Patent Application Publication
(10) Pub. No.: US 2002/0197231 A1
(43) Pub. Date: Dec. 26, 2002

(54) Relaxing gelling bath compositions

(75) Inventors: Florence L'Alloret, Paris (FR); Florence Tournilhac, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/121,865

(22) Filed: Apr. 15, 2002

(30) Foreign Application Priority Data
Apr. 13, 2001 (FR) 01 05112

(51) Int. Cl. A61K 31/74

(52) U.S. Cl. 424/78.02

(57) ABSTRACT

Relaxing gelling bath composition containing at least one gelling polymer or compound, said gelling polymer or compound being a heat-gelling polymer or compound with gelling properties on heating which are reversible with temperature. Use of this polymer or compound for gelling a volume or a mass of water at a predetermined temperature in a receptacle and fluidizing said volume of water when it is cooled, in order to facilitate the discharge from the said receptacle, such as a bathtub.

### BACKGROUND OF THE INVENTION

1. Field of the Invention

The present invention relates to relaxing gelling bath compositions which are optionally foaming, and methods of forming such compositions.

The invention also relates to the use of specific polymers and compounds in these, preferably foaming, relaxing and (heat-)gelling bath compositions.

2. Discussion of the Background

The compositions according to the invention are compositions useful for topical application and in particular for cosmetic or dermatological application.

The technical field of the invention can be defined generally as that of gelling and foaming baths.

There are in fact currently products to be placed in the bath which are both gelling and foaming, such as, for example, the Japanese product sold under the name of Esthe Jelly®. The baths thus thickened provide an extremely relaxing feeling of weightlessness.

Such compositions are disclosed, for example, in the following documents.

The document JP-A-61(86)-68411 discloses a process for applying and absorbing an active substance on a human body, in which a powder of a resin having a high water absorption capacity is added to water or to hot water in a bathtub, as a result of which the water is converted into a fluid aqueous aggregate of particles. An active ingredient is subsequently added and is held uniformly in the spaces between the granules or particles of the aggregate of particles. The active substance can be an oily cosmetic, a nutrient, a vitamin or a pharmaceutical compound.

The resin which has a high water absorption capacity is preferably a polymer compound of high molecular mass comprising a large number of carboxyl and hydroxyl groups. When a powder of such a resin is placed in water, its granules absorb water, swell and take the form of spherical aqueous particles which, even when they are in contact with one another, do not coagulate but instead slide freely over one another to give a fluid aqueous aggregate of particles.

A preferred polymer is Igetagel® P, which would be a copolymer of vinyl alcohol, of vinyl acetate, of acrylic acid and of methyl acrylate in which the carboxyl groups of the acrylic acid have been converted into their sodium salts. Such a pulverulent resin has a water absorbtion capacity of 400 to 500 times its own weight.

The document JP-A-62(87)-106669 discloses a bath product comprising a compound of high molecular mass with a high water absorption capacity as essential ingredient. The compound is chosen from resins prepared by polymerization of a product of polyacrylic acid neutralized with sodium hydroxide, a three-dimensionally crosslinked product of sodium acrylate, a compound prepared by polymerization by grafting of acrylonitrile to starch, and an acrylic acid-vinyl alcohol copolymer.

These compounds absorb water in a proportion of 300 to 1000 times their own weight and solidify in the form of a gel. When they are dissolved in water or hot water, for example the water or hot water of a bathtub, in an amount of 1000 to 2000 times their own weight, they then take the form of a semi-fluid viscous jelly.

The document JP-A-6(94)-172155 relates to a bath product comprising at least one thickener chosen from polysaccharides and their derivatives, water-soluble synthetic polymers and clayey materials capable of expanding in water. The viscosity of the water in which this thickener is dissolved in a proportion of 0.5 weight % vol. is 10 cPs or more.

The synthetic polymers are chosen from carboxyvinyl polymers and their salts, polyvinyl alcohol, polyvinylpyrrolidone, poly(methyl methacrylate), polyacrylic acid neutralized with sodium hydroxide, polyacrylamide and poly(ethylene oxide).

When used in a bath, the product of this document makes it possible to maintain its temperature and that of the body after bathing, and to obtain a uniform temperature throughout the bath.

The upper limit of the viscosity should not exceed 1 000 000 cPs, as, beyond this value, the problems related to cleaning the bathtub become insurmountable.

It emerges from the above that the existing relaxing gelled bath compositions exhibit the major disadvantage of being difficult to remove after bathing, due in particular to their high viscosity, both at the high temperature of the bath and at a lower temperature, following cooling of the latter. A solution which has been provided for overcoming this problem consists in adding salt, in order to fluidize the bath, but the amounts of salt necessary are high, which makes the solution rather impractical to implement.

### OBJECTS OF THE INVENTION

There thus exists a need for a bath composition, that is to say for a composition to be put in a bath, which, first, gels under the conditions of use, namely, generally, at a temperature of the water of 30 to 50° C., and which, secondly, makes possible easy discharge or emptying, for example of the bathtub, after bathing, this discharge generally taking place after the bath has cooled.

In addition, such a composition should preferably be not very aggressive to the skin and should form a stable foam.

### SUMMARY OF THE INVENTION

The aim of the present invention is, inter alia, to meet these needs.

The aim of the present invention is also to provide a heat-gelling bath composition which has relaxing properties, which does not exhibit the inconveniences, limitations, failings and disadvantages of the compositions of the prior art, and which solves the problems of the compositions of the prior art.

This aim and yet others are achieved, in accordance with the invention, by a gelling bath composition comprising at least one gelling polymer or compound, said gelling polymer or compound being a heat-gelling polymer or compound with gelling properties on heating which are reversible with temperature.

According to a preferred embodiment of the invention, the composition is a foaming composition.

In addition, it can comprise one or more surfactants, preferably foaming surfactants.

### DETAILED DESCRIPTION OF THE INVENTION

The polymer or compound included in the bath composition according to the invention first contributes, to this composition, its gelled state under the conditions of use, that is to say at the generally relatively high temperature, for example corresponding to the normal temperature of a bath, and, secondly, makes possible the easy removal of the composition after bathing by simple cooling.

More specifically, above a certain temperature, generally known as the gelling temperature, the polymer or compound, and for this reason the composition, exists in the form of a gel and, below this temperature, the polymer or compound, and therefore the composition, has a markedly lower viscosity and the composition is then generally liquid and therefore easy to discharge.

Any heat-gelling compound or polymer exhibiting gelling properties on heating which are reversible with temperature, particularly that gels at about 30° and above, and reverses below, is suitable for use in the composition of the invention.

These compounds or polymers include the following compounds or polymers:

polymers comprising water-soluble units and units exhibiting, in water, a lower critical solution temperature (LCST);

cellulose derivatives, such as methylcellulose and hydroxypropylmethylcellulose;

copolymers with blocks of polyoxyalkylene type;

nonionic cellulose ethers;

associative water-soluble polymers in the presence of at least one surface-active agent which forms bilayer structures in aqueous solution.

Among these compounds or polymers, preference is given to polymers comprising water-soluble units and units exhibiting, in water, a lower critical solution temperature LCST, as they make it possible to obtain, at low concentrations, transparent heat-gelling aqueous compositions which are less sensitive to the presence of surfactants than compositions formed from other compounds or polymers.

The demixing temperature on heating, in aqueous solution, of the said LCST units of the latter polymers is preferably from 5 to 40° C. for a concentration by mass in water of the said units of 1%.

Surprisingly, it has been shown that the gelling bath compositions according to the invention, which comprise the specific polymer or compound as defined above, make it possible to satisfy the needs listed above and to meet all the requirements mentioned in the preceding part.

In particular, the polymer or compound makes it possible to obtain a bath composition which gels the bath under the normal conditions of use, namely at a temperature of the water generally of 30 to 50° C., but which, surprisingly, makes it possible, after bathing, to easily discharge or empty the receptacle, such as a bathtub, in which the bath is found, by simple cooling.

In other words, after bathing, that is to say when the contents of the receptacle in which bathing has taken place have cooled, the discharge or emptying of the receptacle is easy as the viscosity of the bath, because of the presence of the specific polymer or compound according to the invention, is reduced at these colder temperatures, for example of 5 to 30° C. By way of example, the viscosity is then from 0.001 to 0.5 Pa.s., which corresponds to a fluid or virtually fluid state of easy flow.

Easy or facile discharge or emptying is understood to mean generally that the contents of the receptacle, such as the bathtub, are fluid and low in viscosity and can flow without external intervention and under the effect of gravity alone in the discharge or emptying orifice or orifices normally provided for this purpose.

In contrast to the compositions of the prior art, because of the properties of the specific polymer or compound included in the compositions of the invention, a decrease in the viscosity of the contents of the bath receptacle occurs during the cooling of the bath, whereas, with the compositions of the prior art, the viscosity of the bath remains constant and high, whatever the temperature, which renders the discharge or emptying of the receptacle in which bathing has taken place very difficult, indeed even impossible.

Furthermore, to make possible easy discharge or emptying, in contrast to the compositions of the prior art, it is not necessary for the composition according to the invention to comprise salt and in particular large amounts of salts, since the decrease in the viscosity is inherently due to the properties of the polymer compound.

In addition, the compositions according to the invention can foam while generally only comprising very little or nothing in the way of surfactants.

For this reason, the bath compositions or formulas according to the invention are highly innocuous with respect to the skin.

The (heat-)gelling bath compositions according to the invention generally have high foaming properties and produce a stable foam, in particular above 30° C. and in particular in the presence of hot water at from 30 to 50° C.

It should be noted that a bath composition, according to the invention, is meant to mean a composition to be put in the bath, a composition to be added to a bath, that is to say to an amount of water present or to be put in a receptacle, such as a bathtub. The addition of the composition according to the invention to the water which is present in the said receptacle, which is generally at a temperature greater than the gelling temperature of the polymer or compound, that is to say, for example, from 30 to 50° C., will foam a structure of foam-gel type in the said receptacle. The consistency of such a form-gel is very relaxing. Order of addition of gel and water can be switched.

For this reason, the amount of bath composition according to the invention to be added to the contents of a receptacle for bathing is such that an increase in the viscosity of these contents, that is to say, essentially of the water, is obtained, so as to achieve the appropriate consistency, which can, for example, correspond to a viscosity of greater than or equal to 0.5 Pa.s, and, for example, from 0.5 to 100 Pa.s including 1, 5, 10, 20, 30, 40, 50, 60, 70, 80 and 90 Pa.s.

During cooling after bathing, including simple waiting while the water naturally cools to ambient temperature, the temperature becomes lower than the gelling temperature (Tgel) of the polymer or compound and the bath has, as a result of the reversible heat-gelling properties of the polymers or compounds employed according to the invention, by comparison a much reduced viscosity, which gives it a fluid consistency.

The incorporation of compounds or polymers with reversible heat-gelling properties in bath compositions is neither disclosed nor suggested in the prior art.

The (heat-)gelling bath compositions according to the invention can exist in any form at an ambient temperature during their storage, before being introduced into, mixed with, contacted with, etc. the mass of hot water forming the bath which is at a temperature which is generally greater than their gelling temperature.

This means that, “in the jar”, that is to say before they are mixed with the water of the bath, and prior to the increase in temperature which generally takes place on this occasion, and prior to the possible formation of foam which likewise generally takes place at the time of this use and which is generally related to the agitation of the water in the bath, the range of the textures accessible by the compositions of the invention is unlimited.

The bath compositions according to the invention can exist in the form of solid compositions, preferably as particles or powder, or else in the form of liquid compositions which are optionally gelled, for example of concentrated aqueous solutions.

According to the invention, the composition is preferably a solid composition, in the form of particles: the size and the shape of such particles is unlimited.

They can, for example, be beads, granules, a powder or other particles.

It is possible, for example, to have particles having a particle size ranging from 1 μm to 5×10³ μm.

It is possible to vary at will the form of the composition of the invention and, however, whatever this form, during use, that is to say the incorporation in the water forming the bath, which is accompanied by an increase in temperature (for example, from 30 to 50° C.), the gelling power of the polymer or compound will appear and gelling will occur with possible formation of a stable and lasting foam. This texture disappears during cooling of the bath and the latter becomes low in viscosity and can be easily discharged or emptied.

Furthermore, it has been found that the polymers or compounds included in the composition of the invention, in particular those comprising water-soluble units and LCST units, “do not kill” the foam and that the latter is stable over a long period of time.

The invention additionally relates to the use of heat-gelling polymers or compounds with gelling properties on heating which are reversible with temperature, as described in the present description, for gelling a volume or a mass of water at a predetermined temperature in a receptacle and fluidizing the said volume of water when it has cooled, in order to facilitate the discharge from the said receptacle.

The said predetermined temperature is generally preferably from 30 to 50° C., which commonly corresponds to that of a bath, while the temperature after cooling is generally from 5 to 30° C.

The essential constituent of the compositions according to the invention is a heat-gelling compound or polymer with gelling properties which are reversible with temperature (thermo-reversible).

These polymers or compounds include the following compounds:

water-soluble polymers composed of water-soluble units and units exhibiting, in water, a lower critical solution temperature (LCST);

cellulose derivatives, such as methylcellulose and hydroxypropylmethylcellulose, which, in aqueous solution at certain concentrations (for example, in the region of 3%), exhibit heat-gelling properties within a narrow temperature range, below their lower critical solution temperature (or LCST). Such derivatives are disclosed, for example, in the document N. Sarkar, J. Appl. Polym, Sci., 1979, 24, 1 073;

block copolymers of polyoxyalkylene type (for example of 2 to 6 C), such as Pluronics® (PEO/PPO/PEO), which exhibit, at concentrations of the order of 20 to 30% in aqueous solution, a sol/gel transition on raising the temperature, as is mentioned in the document W. Brown, K. Schillen, M. Almgren, S. Hvidt, P. Bahadur, J. Phys. Chem., 1991, 95, 1 850. This gelling on heating is reversible with temperature;

nonionic cellulose ethers, such as ethylhydroxyethyl cellulose, which, in aqueous solution, show, in the presence of ionic surfactants (such as sodium dodecyl sulphate), a thickening power when the temperature increases, as is described in the document A. Carlsson, G. Karlstrom, B. Lindman, Coll. Surf., 1990, 47, 147;

associative water-soluble polymers, in the presence of at least one surface-active agent which forms bilayer structures in aqueous solution, described in the document K. Loyen, I. Iliopoulos, R. Audebert, Langmuir, 1994, 10, 1 421. For example, a polyacrylic acid with a molar mass of 150 000 g/mol, modified with 3% (in moles) of octadecyl chain, exhibits, as a 1% (by weight) aqueous solution, in the presence of 0.1 mol/l of the surfactant C₁₂(EO)₄, gelling properties on raising the temperature.

Preference is given, among reversible heat-gelling systems, to water-soluble polymers composed of water-soluble units and units exhibiting, in water, a lower critical solution temperature (LCST), as they make it possible to obtain, at low concentrations (a few per cent), transparent heat-gelling aqueous compositions which are less sensitive to the presence of surfactants than compositions formed from the other polymers or compounds described.

In this respect, it is useful to point out that LCST units is understood to mean units with a solubility in water which is modified above a certain temperature. They are units which exhibit a demixing temperature on heating (or cloud point) defining their region of solubility in water. The minimum demixing temperature obtained as a function of the concentration of polymer composed solely of LCST units is known as the LCST (Lower Critical Solution Temperature). For each concentration of LCST polymer, a demixing temperature on heating is observed. It is greater than the LCST, which is the minimum point on the curve. Below this temperature, the polymer is soluble in water, above this temperature, the polymer loses its solubility in water.

The LCST units of the preferred polymer used in the composition according to the invention preferably correspond to a specific definition proportions of the LCST units in the polymer of 5 to 70%, preferably of 20 to 65%, and gelling temperature of the polymer of 5 to 50° C. for a concentration by mass of 2% by weight in water) which, essentially, makes it possible to give the polymer comprising them the advantageous heat-gelling properties as claimed above.

The LCST units of the preferred polymer have, according to the invention, a solution temperature on heating of 5 to 50° C. for a concentration by mass in water of 1% by weight of the said LCST units.

Preferably, the solution temperature on heating in aqueous solution of the LCST units of the preferred polymer is from 15 to 40° C. and more preferably from 20 to 35° C., for a concentration by mass in water of 1% of the said LCST units.

Preferably, the concentration of the polymer or compound, in particular of the preferred polymer, in the final bath prepared from the “bath composition”, that is to say from the composition to be put in the bath according to the invention, is such that the gelling temperature of the bath is within the range from 20 to 35° C.

The concentration of the polymer or compound, in particular of the preferred polymer, in the final bath will generally be from 1 to 200 g/l in order to obtain the desired texture at the usual temperature of a bath, which is generally from 30 to 50° C.

The preferred polymer included in the bath composition, having the structure described above with water-soluble units and specific LCST units defined above, exhibits, in aqueous solution, gelling properties above a critical temperature or heat-gelling properties.

These gelling properties on heating observed above the demixing temperature of the LCST chains are described in the prior art, in particular in the documents D. Hourdet et al., Polymer, 1994, Vol. 35, No. 12, pp. 2 624-2 630, F. L'Alloret et al., Coll. Polym. Sci., 1995, Vol. 273, pp. 1 163-1 173 and F. L'Alloret, Revue de l'Institut Français du Pétrole, 1997, Vol. 52, No. 2, pp. 117-128. They are due to the association of the LCST chains within hydrophobic microdomains above their solution temperature, thus forming nodes for crosslinking between the main chains.

These gelling properties are observed when the concentration of preferred polymer is sufficient to make possible interactions between LCST grafts carried by different macromolecules. The minimum concentration necessary, known as the critical aggregation concentration or CAC, is evaluated by rheology measurements: it is the concentration above which the viscosity of an aqueous solution of the preferred polymers included in the compositions of the invention becomes greater than the viscosity of a solution of the equivalent polymer not comprising LCST chains.

Above the CAC, the preferred polymers of the invention exhibit gelling properties when the temperature becomes greater than a critical value known as the gelling temperature or Tgel. According to the data in the literature, good agreement exists between Tgel and the demixing temperature of the LCST chains under the same concentration conditions. The gelling temperature of an aqueous solution of a preferred polymer included in the composition of the invention is determined by rheology measurements: it is the temperature above which the viscosity of the solution of a preferred polymer included in the composition of the invention becomes greater than the viscosity of a solution of the equivalent polymer not comprising LCST chains.

The preferred polymers of the invention are characterized by a specific gelling temperature generally of 5 to 50° C., preferably of 15 to 40° C., for a concentration by mass in water for example equal to 2% by weight.

The other polymers and compounds included in the compositions of the invention have gelling temperatures of 15 to 50° C. for a concentration by mass in water for example of 0.1 to 50% by weight.

This specific gelling temperature allows these polymers and compounds, in particular the preferred polymers, to give the compositions of the invention all the advantageous properties cited above and in particular their gelled foam texture at the “hot” temperature of the bath and the fluidity when cooled.

Heat-gelling polymers or compounds with gelling properties which are reversible with temperature are known from the prior art but their use in baths, in order in particular to facilitate the discharge therefrom, is never described nor suggested.

In particular, polymers comprising, after the fashion of those preferably employed in the compositions of the invention, water-soluble units and LCST units and having gelling properties on heating observed above the demixing temperature of the LCST chains are also described in the documents already cited above.

The document D. Hourdet et al., Polymer, 1994, Vol. 35, No. 12, pp. 2 624-2 630 relates to the reversible heat-induced thickening of aqueous solutions of a copolymer comprising a water-soluble polyacrylic acid backbone with poly(ethylene oxide) (PEO) grafts.

The document F. L'Alloret et al., Coll. Polym. Sci., 1995, Vol. 273, pp. 1 163-1 173 relates to the heat-induced thickening behaviour in aqueous solution of polymers comprising a 2-acrylamido-2-methylpropane sulphonic acid (AMPS) backbone and poly(ethylene oxide) side chains.

Likewise, the document F. L'Alloret, Revue de l'Institut Français du Pétrole, 1997, Vol. 52, No. 2, pp. 117-128 describes the reversible heat-induced association of copolymers with a water-soluble polyacrylic backbone or based on AMPS with PEO grafts.

Polymers such as those mentioned in the documents D. Hourdet et al., Polymer, 1994, Vol. 35, No. 12, pp. 2 624-2 630, F. L'Alloret et al., Coll. Polym. Sci., 1995, Vol. 273, pp. 1 163-1 173 and F. L'Alloret, Revue de l'Institut Français du Pétrole, 1997, Vol. 52, No. 2, pp. 117-128 are used in particular in the oil industry.

Thus, the document EP-A-0 583 814 discloses heat-viscosifying polymers with a water-soluble backbone comprising LCST segments, or carrying LCST side chains, which can be used in particular as thickening agents, constituents of drilling fluids or other fluids, and industrial cleaning fluids.

The document EP-A-0 629 649 discloses polymers analogous to those of the document EP-A-0 583 814 and their use as antisedimentation agent for suspensions, optionally in cosmetic preparations.

It should be noted that none of the documents described above mentions the incorporation of the polymers in gelling bath compositions and that, in addition, the polymers described do not comprise the specific LCST units of the preferred polymers according to the invention.

The document WO-A-95 24430 also discloses copolymers comprising a backbone composed of pH-sensitive units, for example polyacrylic units, and temperature-sensitive units grafted to this backbone. These copolymers exhibit temperature gelling properties and they are used for the liberation and the controlled release of active or pharmaceutical and optionally cosmetic principles by topical application. The use of these polymers in bath products is not mentioned.

The temperature-sensitive units of the copolymers of this document are not the specific LCST units of the preferred polymers of the invention. Furthermore, the polymers according to the document WO-A-95 24430 are characterized by the highly troublesome temperature opacity of the products obtained, which is not the case with the preferred polymers employed in the compositions of the invention.

In fact, the polymer of this document is fundamentally different from the preferred polymer of the invention as it exhibits overall, for the entire polymer, an LCST within the temperature range from 20 to 40° C., in contrast to the polymers of the invention, which are water-soluble at any temperature.

The documents U.S. Pat. No. 5,939,485 and WO-A-97 00275 disclose polymer systems with reversible gelling, comprising a sensitive component capable of aggregating, in response to a change in an external “stimulus”, and a structural component. The external stimulus can be, for example, the temperature. Thus, it is indicated that an increase in the viscosity by at least a factor of 30 is obtained for an aqueous solution of polymer at a concentration by mass of less than 4%.

The component sensitive to the external stimulus is fundamentally different from the LCST units of the preferred polymers of the application. This is because these components sensitive to the external stimulus are in fact composed of at least one hydrophilic fragment and one hydrophobic fragment. Thus, the sensitive component can be a block copolymer, such as a poloxamer, for example a Pluronic®, which is a block polymer of ethylene oxide (soluble) and of propylene oxide (insoluble); such a block copolymer aggregates at the microscopic level above a critical temperature not corresponding to an LCST. A nonionic surfactant can also be used as sensitive component.

The document U.S. Pat. No. 5,939,485 relates more particularly to a polymer network formed from a water-soluble polyacrylic backbone and from a sensitive Pluronic® component, which is entangled in the said backbone without covalent bonding; this network therefore has a specific structure which has nothing in common with the preferred polymer of the invention. In contrast, the document WO-A-97 00275 gives polymers with covalent bonds.

These polymers have gelling properties on heating and they can be used in the pharmaceutical field for the delivery of medicaments and in numerous other fields, including the field of cosmetics. As regards the cosmetic applications mentioned, only emulsion examples are mentioned. They all comprise a neutral, anionic or cationic surfactant, in addition to the polymer acting as gelling agent.

The use of the polymers in gelling baths is never mentioned, nor in particular the easy emptying effects on cooling obtained in the invention.

In these formulations, the sensitive component of the polymer system behaves differently from LCST units, such as those of the preferred polymer of the invention, during heating. Thus, when the said sensitive component (for example polyoxamer) is heated to approximately 30-40° C., it exhibits a micellization, that is to say an aggregation on the list microscopic scale, temperature and then, when it is heated further, a much higher LCST temperature. This LCST corresponds to an aggregation on the macroscopic scale between the macromolecules. It is explained in WO-A-97 00275, on pages 16 and 17, that the gelling and the LCST are observed at temperatures which differ by approximately 70° C., the gelling temperature corresponding to the micellization temperature of the sensitive component, which shows that these polymers are different from those, which are preferred, of our application. In addition, it is not possible, because of the synthesis used in the document WO-A-97 00275, to filly control the structure and the properties of the final polymer obtained, as is the case in the compositions of the invention, in particular for the preferred polymers.

Cosmetic compositions using a polymer system with reversible heat-induced gelling, comprising polyacrylic acid and a poloxamer as in the documents U.S. Pat. No. 5,939,485 and WO-A-97 00275, are also known from the document WO-A-98 48768. Again, the polymer system of these documents is fundamentally different from those employed in the compositions of the invention, with the result that the advantageous properties of the composition of the invention cannot be obtained.

WO-A-00 35961 discloses the preparation of polymers having temperature-induced thickening properties by emulsion polymerization and the use of these polymers in pharmaceutical and cosmetic compositions. These polymers can be copolymers having water-soluble units and LCST units based on alkylene oxide. Provision is made for the addition of nonionic surfactants to the polymers to strengthen their heat-induced thickening properties.

It emerges from the above that the use in foaming, gelling bath compositions of the polymers or compounds according to the invention, and in particular the use of the preferred polymers according to the invention exhibiting specific LCST units, conferring on these baths gelling properties at the temperature of use which go hand in hand with a reduced viscosity under the effect of cooling, is neither described nor suggested in the documents of the prior art.

The preferred polymers (comprising water-soluble units and comprising LCST units) used in the invention can be block polymers or graft polymers which comprise, on the one hand, water-soluble units and, on the other hand, LCST units as defined above.

It is specified that, in the present text, the water-soluble units or the LCST units of the preferred polymers employed according to the invention are defined as not including the groups which bond to one another, on the one hand, the said water-soluble units and, on the other hand, the said LCST units.

The said bonding groups result from the reaction, during the preparation of the preferred polymer, of reactive sites carried, on the one hand, by the precursors of the said water-soluble units and, on the other hand, by the precursors of the said LCST units.

The preferred polymers employed in the context of the invention can therefore be block polymers comprising, for example, blocks composed of water-soluble units alternating with LCST blocks.

These polymers can also exist in the form of graft polymers, the backbone of which is formed from water-soluble units, the said backbone carrying grafts composed of LCST units.

The said polymers can be partially crosslinked.

The term “water-soluble units” is generally understood to mean that these units are units which are soluble in water, at a temperature from 5 to 80° C., in a proportion of at least 10 g/l, preferably of at least 20 g/l.

However, the term “water-soluble units” is also understood to mean units not necessarily having the above mentioned solubility but which, as a 1% by weight aqueous solution, from 5 to 80° C., make it possible to obtain a macroscopically homogeneous and transparent solution, that is to say having a maximum light transmission value, whatever the wavelength between 400 and 800 nm, through a sample with a thickness of 1 cm, of at least 85%, preferably of at least 90%.

These water-soluble units do not exhibit a demixing temperature on heating of LCST type.

These water-soluble units are capable, in all or in part, of being obtained by polymerization, in particular radical polymerization, or by polycondensation or alternatively are composed, in all or in part, of existing natural or modified natural polymers.

By way of example, the water-soluble units are capable, in all or in part, of being obtained by polymerization, in particular radical polymerization, of at least one monomer chosen from the following monomers:

1) (meth)acrylic acid;

2) vinyl monomers of following formula (I):

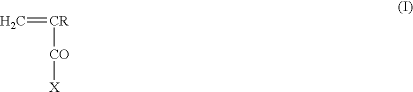

in which:

R is chosen from H, —CH₃, —C₂H₅ or —C₃H₇; and

X is chosen from:

alkyl oxides of —OR′ type, where R′ is a saturated or unsaturated and linear or branched hydrocarbon-based radical having from 1 to 6 carbon atoms, optionally substituted by at least one halogen atom (iodine, bromine, chlorine, fluorine); or one group of the following types: sulphonic (—SO₃⁻), sulphate (—SO₄⁻)or phosphate (—PO₄H₂); hydroxyl (—OH); primary amine (—NH₂); or secondary (—NHR₁), tertiary (—NR₁R₂) or quaternary (—N⁺R₁R₂R₃) amine with R₁, R₂ and R₃ being, independently of one another, a saturated or unsaturated and linear or branched hydrocarbon-based radical having 1 to 6 carbon atoms, with the proviso that the sum of the carbon atoms of R′+R₁+R₂+R₃ does not exceed 7; and

—NH₂, —NHR₄ and —NR₄R₅ groups in which R₄ and R₅ are, independently of one another, saturated or unsaturated and linear or branched hydrocarbon-based radicals having 1 to 6 carbon atoms, with the proviso that the total number of carbon atoms of R₄+R₅ does not exceed 7, the said R₄ and R₅ radicals optionally being substituted by a halogen atom (iodine, bromine, chlorine, fluorine); or a group of the following types: hydroxyl (—OH); sulphonic (—SO₃⁻); sulphate (—SO₄⁻); phosphate (—PO₄H₂); primary amine (—NH₂); or secondary (—NHR₁), tertiary (—NR₁R₂) and/or quaternary (—N⁺R₁R₂R₃) amine with R₁, R₂ and R₃ being, independently of one another, a saturated or unsaturated and linear or branched hydrocarbon-based radical having 1 to 6 carbon atoms, with the proviso that the sum of the carbon atoms of R₄+R₅+R₁+R₂+R₃ does not exceed 7;

mention may in particular be made, as vinyl monomer of formula (I), of 2-acrylamido-2-methylpropanesulphonic acid (AMPS) or (meth)acrylic acid.

3) maleic anhydride;

4) itaconic acid;

5) vinyl alcohol of formula CH₂═CHOH;

6) vinyl acetate of formula CH₂═CH—OCOCH₃;

7) N-vinyllactams, such as N-vinylpyrrolidone, N-vinylcaprolactam and N-butyrolactam;

8) vinyl ethers of formula CH₂═CHOR₆, in which R₆ is a saturated or unsaturated and linear or branched hydrocarbon-based radical having from 1 to 6 carbons;

9) water-soluble styrene derivatives, in particular styrenesulphonate;

10) dimethyldiallylammonium chloride; and

11) vinylacetamide.

The polycondensates and the natural or modified natural polymers which can constitute all or part of the water-soluble units are chosen from one or more of the following components:

water-soluble polyurethanes,

xanthan gum, in particular that sold under the names Keltrol T and Keltrol SF by Kelco; or Rhodigel SM and Rhodigel 200 from Rhodia;

alginates (Kelcosol from Monsanto) and their derivatives, such as propylene glycol alginate (Kelcoloid LVF from Kelco);

cellulose derivatives and in particular carboxymethylcellulose (Aquasorb A500, Hercules), hydroxypropylcellulose, hydroxyethylcellulose and quaternized hydroxyethylcellulose;

galactomannans and their derivatives, such as konjac gum, guar gum, hydroxypropylguar, hydroxypropylguar modified by sodium methylcarboxylate groups (Jaguar XC97-1, Rhodia), or guar hydroxypropyltrimethylammonium chloride.

Mention may also be made of polyethyleneimine.

Preferably, the water-soluble units have a molar mass ranging from 1 000 g/mol to 50 000 000 g/mol when they constitute the water-soluble backbone of a graft polymer.

These water-soluble units preferably have a molar mass ranging from 500 g/mol to 100 000 g/mol when they constitute a block of a multiblock polymer.

Preferably, the water-soluble units comprise at least one monomer chosen from (meth)acrylic acid and 2-arylamido-2-methylpropanesulphonic acid.

The LCST units of the polymers, in particular of the preferred polymers used in the invention, can be defined as being units with a solubility in water which is modified above a certain temperature. They are units exhibiting a demixing temperature on heating (or cloud point) defining their regions of solubility in water. The minimum demixing temperature obtained as a function of the concentration of polymer is known as the LCST (Lower Critical Solution Temperature). For each concentration of polymer, a demixing temperature on heating is observed; it is greater than the LCST, which is the minimum point on the curve. Below this temperature, the polymer constituting the LCST unit is soluble in water; above this temperature, the polymer constituting the LCST unit loses its solubility in water.

Some of these LCST polymers are described in particular in the articles by Taylor et al., Journal of Polymer Science, part A: Polymer Chemistry, 1975, 13, 2 551 [15]; J. Bailey et al., Journal of Applied Polymer Science, 1959, 1, 56 [16]; and by Heskins et al., Journal of Macromolecular Science, Chemistry A2, 1968, Vol. 8, 1 441 [17].

Soluble in water at the temperature T is understood to mean that the units exhibit a solubility at T of at least 1 g/l, preferably of at least 2 g/l.

The LCST can be measured visually: the temperature at which the cloud point of the aqueous solution appears is determined; this cloud point is reflected by the opacification of the solution or loss of transparency.

Generally, a transparent composition will have a maximum light transmission value, whatever the wavelength between 400 and 800 nm, through a sample with a thickness of 1 cm, of at least 85%, preferably of at least 90%.

The transmission can be measured by placing a sample with a thickness of 1 cm in the light beam of a spectrophotometer operating in the wavelengths of the visible spectrum.

The LCST units of the preferred polymers used in the invention can be composed of one or more polymers chosen from the following polymers:

polyethers, such as poly(ethylene oxide) (PEO), polypropylene oxide) (PPO) or random copolymers of ethylene oxide (EO) and of propylene oxide (PO);

poly(vinyl methyl ether)s,

N-substituted polymeric and copolymeric acrylamide derivatives having an LCST, such as poly-N-isopropylacrylamide (Nipam) and poly-N-ethylacrylamide, and

poly-N-vinylcaprolactam and copolymers of N-vinylcaprolactam.

Preferably, the LCST units are composed of poly(propylene oxide) (PO)ₙ, where n is an integer from 10 to 50, or of random copolymers of ethylene oxide (EO) and of propylene oxide (PO), represented by the formula:

(EO)ₘ(PO)ₙ

in which m is an integer ranging from 1 to 40, preferably from 2 to 20, and n is an integer ranging from 10 to 60, preferably from 20 to 50.

Preferably, the molar mass of these LCST units is from 500 to 5 300 g/mol, more preferably from 1 500 to 4 000 g/mol.

It may be found that the random distribution of the EO and PO units is reflected by the existence of a lower critical solution temperature above which macroscopic phase separation is observed. This behaviour is different from that of (EO)(PO) block copolymers, which micellize above what is known as a critical micellization temperature (aggregation on the microscopic scale).

The LCST units can therefore in particular be random copolymers of ethylene oxide and of propylene oxide which are aminated, in particular monoaminated, diaminated or triaminated. These polymers, before reaction, carry reactive sites, in this case amino groups, which react with the reactive sites of the water-soluble polymers, for example carboxyl groups, to give the final polymer employed in the invention. In the final polymer, water-soluble units are bonded to the LCST units via bonding groups resulting from the reaction of the reactive groups or sites carried respectively by the LCST units and the precursors of the water-soluble units. These bonding groups will, for example, be amide, ester, ether or urethane groups.

Mention may be made, among these commercially available LCST polymers, of the copolymers sold under the name Jeffamine by Huntsman and in particular Jeffamine XTJ-507 (M-2005), Jeffamine D-2000 and Jeffamine XTJ-509 (or T-3000).

The LCST units can also result from random EO/PO copolymers with OH ends, such as those sold under the name Polyglycol P41 and Polyglycol B11 by Clariant.

Use may also be made, in the preferred polymers of the invention, as LCST units, of N-substituted polymeric and copolymeric acrylamide derivatives having an LCST, and of poly-N-vinylcaprolactam and copolymers of N-vinylcaprolactam.

Mention may be made, as examples of N-substituted polymeric and copolymeric acrylamide derivatives having an LCST, of poly-N-isopropylacrylamide, poly-N-ethylacrylamide and copolymers of N-isopropylacrylamide (or of N-ethylacrylamide) and of a vinyl monomer chosen from monomers having the formula (I) given above, maleic anhydride, itaconic acid, vinylpyrrolidone, styrene and its derivatives, dimethyldiallylammonium chloride, vinylacetamide, vinyl alcohol, vinyl acetate, vinyl ethers and vinyl acetate derivatives.

The molar mass of these polymers is preferably from 1 000 g/mol to 500 000 g/mol, preferably from 2 000 to 50 000 g/mol.

These polymers can be synthesized by radical polymerization using a pair of initiators, such as aminoethanethiol hydrochloride, in the presence of potassium persulphate, in order to obtain precursor oligomers having an aminated reactive end.

Mention may be made, as examples of N-vinylcaprolactam copolymers, of the copolymers of N-vinylcaprolactam and of a vinyl monomer having the formula (I) given above or of a monomer chosen from maleic anhydride, itaconic acid, N-vinylpyrrolidone, styrene and its derivatives, dimethyldiallylammonium chloride, vinylacetamide, vinyl alcohol, vinyl acetate, vinyl ethers and vinyl acetate derivatives.

The molar mass of these vinylcaprolactam polymers or copolymers is generally from 1 000 g/mol to 500 000 g/mol, preferably from 2 000 to 50 000 g/mol.

These compounds can be synthesized by radical polymerization using a pair of initiators, such as aminoethanethiol hydrochloride, in the presence of potassium persulphate, in order to obtain LCST units having an aminated reactive end.

The proportion by mass of the LCST units in the final polymer is preferably from 5% to 70%, in particular from 20% to 65% and particularly from 30% to 60% by weight, with respect to the final polymer.

It was seen above that the demixing temperature on heating of said LCST units of the preferred polymer used in the invention is from 5 to 50° C., preferably from 15 to 40° C., for a concentration by mass in water of 1% by weight of said LCST units.

The preferred polymers employed in the context of the invention can be easily prepared by a person skilled in the art on the basis of his general knowledge, using grafting, copolymerization or coupling reaction processes.

When the final polymer exists in the form of a graft polymer, exhibiting in particular a water-soluble backbone with LCST side chains or grafts, it is possible to prepare it by grafting LCST units having at least one reactive end or reactive site, in particular aminated end(s) or site(s), to a water-soluble polymer which forms the backbone and which carries a minimum of 10% (in moles) of reactive groups, such as carboxylic acid functional groups. This reaction can be carried out in the presence of a carbodiimide, such as dicyclohexylcarbodiimide or 1-(3-dimethylaminopropyl)-3-ethylcarbodiimide hydrochloride, in a solvent, such as N-methylpyrrolidone or water.

Another possibility for preparing graft polymers consists in copolymerizing, for example, an LCST macromonomer (LCST chain described above with a vinyl end) and a water-soluble vinyl monomer, such as acrylic acid or vinyl monomers having the formula (1), such as 2-acrylamido-2-methylpropanesulphonic acid (AMPS).

When the final polymer exists in the form of a block polymer, it is possible to prepare it by coupling between water-soluble units and LCST units, these units having, at each end, complementary reactive sites.

In the case of grafting processes and coupling processes, reactive sites of the LCST units can be amine function groups, in particular monoamine, diamine or triamine functional groups, and OH functional groups. In this case, the reactive sites of the water-soluble units can be carboxylic acid functional groups. The groups bonding the water-soluble units and the LCST units will therefore be, for example, amide groups or ester groups.

As was seen above, the gelling compositions of the invention can be in a form ranging from the liquid form to the solid form (liquid, creamy, pasty or solid form), for example as particles.

The composition, when it is liquid, can be more or less fluid. It essentially comprises a continuous aqueous phase. This continuous aqueous phase generally exists in the form of a concentrated solution, preferably a concentrated aqueous solution, of polymer or compound.

According to the invention, the aqueous phase comprises a heat-gelling polymer or compound with gelling properties which are reversible with temperature. It is preferably a water-soluble polymer comprising water-soluble units and LCST units as defined above.

The solution is generally a concentrated solution, that is to say that the concentration by mass of polymer or compound in the aqueous phase is from 0.1 to 90%, preferably from 0.5 to 90%.

Generally, this concentration should be sufficient to produce the desired gelled structure on adding a limited amount, for example 100 g, of the composition according to the invention to a bath with a standard volume, for example 200 1.

When the composition according to the invention is a solid composition, for example a particulate composition and in particular a pulverulant composition, the concentration by mass of the polymer or compound lies within similar ranges, namely from 0.5 to 1 g of polymer or compound per g of solid composition, for example of powder.

The gelling composition according to the invention, by virtue of the presence of the specific polymer or compound described above, can constitute a foaming composition, even with no or little surfactant, that is to say that the concentration of surfactant is, for example, equal to or less than 5% by mass, preferably from 1 to 5%, including 2, 3 and 4%.

When the composition comprises one or more surfactants, according to the final use, it can comprise up to 20% by weight of surfactants, for example from 0.05 to 20% and better still from 0. I to 15% by weight, with respect to the total weight of the composition.

The foaming surfactants which can be used in the composition of the invention can be nonionic, anionic, amphoteric or zwitterionic surfactants and their mixtures; nonionic surfactants being preferred.

Mention may be made, as nonionic surfactants, of, for example, alkylpolyglycosides (APG), esters of polyols and of fatty acids, esters of polyethylene glycols and of fatty acids, derivatives of fatty alcohols and of polyols (ethers), and oxyalkylenated (oxyethylenated and/or oxypropylenated) derivatives of these compounds. Mention may also be made of maltose esters, polyglycerolated fatty alcohols, glucamine derivatives, such as 2-ethylhexyloxycarbonyl-N-methylglucamine, and their mixtures.

Mention may in particular be made, as alkylpolyglycosides, of alkylpolyglucosides and, for example, decylglucoside ((C₉/C₁₁ alkyl)polyglycoside (1,4)), such as the product sold under the name Mydol 10 by Kao Chemicals, the products sold under the name Plantaren 2000 UP and Plantacare 2000 UP by Henkel, and the product sold under the name Oramix NS 10 by Seppic; caprylyl/caprylglucoside, such as the products sold under the name Ormaix CG 110 by Seppic or under the name Lutensol GD 70 by BASF; laurylglucoside, such as the products sold under the names Plantaren 1200 N and Plantacare 1200 by Henkel;

and cocoglucoside, such as the product sold under the name Plantacare 818/UP by Henkel, and their mixtures.

The maltose derivatives are, for example, those disclosed in the document EP-A-0 566 438 [18], such as 6′-(O-octanoyl)-D-maltose, or alternatively 6′-(O-dode-canoyl)-D-maltose, disclosed in the document FR-A-2 739 556 [19].

Mention may be made, among polyglycerated fatty alcohols, of polyglycerated dodecanediol (3.5 mol of glycerol), a product sold under the name Chimexane NF by Chimex.

Use may be made, as anionic surfactants, of, for example, carboxylates, amino acid derivatives, alkyl sulphates, alkyl ether sulphates, sulphonates, isethionates, taurates, sulphosuccinates, alkyl sulphocetates, phosphates and alkyl phosphates, polypeptides, anionic alkylpolyglycoside derivatives, fatty acid soaps, and their mixtures.

Mention may be made, as carboxylates, of, for example, alkaline salts of N-acylamino acids; amido ether carboxylates (AEC), such as sodium lauryl amido ether carboxylate (3 EO), sold under the name Akypo Foam 30 by Kao Chemicals; polyoxyethylene carboxylic acid salts, such as oxyethylenated (6 EO) odium lauryl ether carboxylate (65/25/10 C₁₂-C₁₄-C₁₆), sold under the name Akypo Soft 45 NV by Kao Chemicals; polyoxyethylenated and carboxymethylated olive oil fatty acids, such as the product sold under the name Olivem 400 by Biologia E Tecnologia; oxyethylenated (6 EO) sodium tridecyl ether carboxylate, sold under the name Nikkol ECTD-6NEX by Nikkol; or sodium 2-(2-hydroxyalkyloxy)acetate, sold under the name Beaulight Shaa by Sanyo.

The amino acid derivatives can be chosen, for example, from sarcosinates and in particular acylsarcosinates, such as sodium lauroylsarcosinate, sold under the name Sarkosyl NL 97 by Ciba or sold under the name Oramix L 30 by Seppic, sodium myristoylsarcosinate, sold under the name Nikkol Sarcosinate MN by Nikkol, or sodium palmitoylsarcosinate, sold under the name Nikkol Sarcosinate PN by Nikkol; alaninates, such as sodium N-lauroyl-N-methylamidopropionate, sold under the name Sodium Nikkol Alaninate LN 30 by Nikkol or sold under the name Alanone Ale by Kawaken, and triethanolamine N-lauroyl-N-methylalanine, sold under the name Alanone Alta by Kawaken; N-acylglutamates, such as triethanolamine monococoylglutamate, sold under the name Acylglutamate CT-12 by Ajinomoto, triethanolamine lauroylglutamate, sold under the name Acylglutamate LT-12 by Ajinomoto, and monosodium N-lauroyl-L-glutamate, sold under the name Amisoft LS-11 by Ajinomoto; aspartates, such as the mixture of triethanolamine N-lauroylaspartate and of triethanolamine N-myristoylaspartate sold under the name Asparack LM-TS2 by Mitsubishi; citrates, and their mixtures.

Mention may be made, as glycine derivatives, of sodium N-cocoylglycinate and potassium N-cocoylglycinate, such as the products sold under the names Amilite GCS-12 and Amilite GCK-12 by Ajinomoto.

Mention may be made, as alkyl ether sulphates, of, for example, sodium lauryl ether sulphate (70/30 C₁₂-C₁₄) (2.2 EO), sold under the names Sipon AOS 225 or Texapon N702 Paté by Henkel, ammonium lauryl ether sulphate (70/30 C₁₂-C₁₄) (3 EO), sold under the name Sipon LEA 370 by Henkel, or ammonium (C₁₂-C₁₄)alkyl ether (9 EO) sulphate, sold under the name Rhodapex AB/20 by Rhodia Chimie.

Mention may be made, as sulphonates, of, for example, alpha-olefin sulphonates, such as sodium alpha-olefin sulphonate (C₁₄-C₁₆), sold under the name Bio-Terge AS-40 by Stepan, sold under the names Witconate AOS Protégé and Sulfamine AOS PH 12 by Witco or sold under the name Bio-Terge AS-40 CG by Stepan, secondary sodium olefin sulphonate, sold under the name Hostapur SAS 30 by Clariant; or linear alkylaryl sulphonates, such as sodium xylene sulphonate, sold under the names Manrosol SXS30, Manrosol SXS40 and Manrosol SXS93 by Manro.

Mention may be made, as isethionates, of acylisethionates, such as sodium cocoylisethionate, for example the product sold under the name Jordapon CI P by Jordan.

Mention may be made, as taurates, of the sodium salt of palm kernel oil methyltaurate, sold under the name Hostapon CT Paté by Clariant; N-acyl-N-methyltaurates, such as sodium N-cocoyl-N-methyltaurate, sold under the name Hostapon LT-SF by Clariant or sold under the name Nikkol CMT-30-T by Nikkol, or sodium palmitoyl methyltaurate, sold under the name Nikkol PMT by Nikkol.

Mention may be made, as sulphosuccinates, of, for example, oxyethylenated (3 EO) lauryl (70/30 C₁₂-C₁₄) alcohol monosulphosuccinate, sold under the names Setacin 103 Special and Rewopol SB-FA 30 K 4 by Witco, the disodium salt of a hemisulphosuccinate of C₁₂-C₁₄ alcohols, sold under the name Setacin F Special Paste by Zschimmer Schwarz, oxyethylenated (2 EO) disodium oleamidosulphosuccinate, sold under the name Standapol SH 135 by Henkel, oxyethylenated (5 EO) lauramide monosulphosuccinate, sold under the name Lebon A-5000 by Sanyo, the disodium salt of oxyethylenated (10 EO) lauryl citrate monosulphosuccinate, sold under the name Rewopol SB CS 50 by Witco, or ricinoleic monoethanolamide monosulphosuccinate, sold under the name Rewoderm S 1333 by Witco.

Mention may be made, as phosphates and alkyl phosphates, of, for example, monoalkyl phosphates and dialkyl phosphates, such as lauryl monophosphate, sold under the name MAP 20 by Kao Chemicals, the potassium salt of dodecyl phosphate, a mixture of mono- and diester (predominantly diester) sold under the name Crafol AP-31 by Cognis, the mixture of octyl phosphate monoester and diester, sold under the name Crafol AP-20 by Cognis, the mixture of ethoxylated (7 mol of EO) 2-butyloctyl phosphate monester and diester sold under the name Isofol 12 7 EO-Phosphate Ester by Condea, the potassium or triethanolamine salt of mono(C₁₂-C₁₃)alkyl phosphate, sold under the references Arlatone MAP 230K-40 and Arlatone MAP 230T-60 by Uniqema, or potassium lauryl phosphate, sold under the name Dermalcare MAP XC-99/09 by Rhodia Chimie.

The polypeptides are obtained, for example, by condensation of a fatty chain with cereal amino acids and in particular wheat and oat amino acids. Mention may be made, as polypeptides, of, for example, the potassium salt of hydrolysed lauroyl wheat protein, sold under the name Aminofoam W OR by Croda, the triethanolamine salt of hydrolysed cocoyl soybean protein, sold under the name May-Tein SY by Maybrook, the sodium salt of oat lauroyl amino acids, sold under the name Proteol Oat by Seppic, collagen hydrolysate grafted to coconut fatty acid, sold under the name Geliderm 3000 by Deutsche Gelatine, or soybean proteins acylated with hydrogenated coconut acids, sold under the name Proteol VS 22 by Seppic.

The anionic alkylpolyglycoside derivatives can in particular be citrates, tartrates, sulphosuccinates, carbonates and glycerol ethers obtained from alkylpolyglycosides and in particular polyalkylglucosides. Mention may be made, for example, of the sodium salt of cocoylpolyglycoside (1,4) tartaric ester, sold under the name Eucarol AGE-ET by Cesalpinia, the disodium salt of cocoylpolyglycoside (1,4) sulphosuccinic ester, sold under the name Essai 512 MP by Seppic, or the sodium salt of cocoylpolyglycoside (1,4) citric ester, sold under the name Eucarol AGE-EC® by Cesalpinia. Another anionic polyglycoside derivative can be sodium dodecyl-D-galactosideuronate, sold under the name Dodecyl-D-galactoside Uronate de Sodium by Soliance.

The fatty acid soaps which can be used as anionic surfactants are fatty acids of natural or synthetic origin, salified with an inorganic or organic base. The fatty chain can comprise from 6 to 22 carbon atoms, preferably from 8 to 18 carbon atoms. The inorganic or organic base can be chosen from alkali metals or alkaline earth metals, amino acids and aminoalcohols. Use may be made, as salts, of, for example, sodium, potassium, magnesium, triethanolamine, N-methylglucamine, lysine and arginine salts. Mention may be made, as soaps, of, for example, the potassium or sodium salts of lauric acid, myristic acid, palmitic acid or stearic acid (potassium or sodium laurate, myristate, palmitate and stearate), and their mixtures.

Use may be made, as amphoteric and zwitterionic surfactants, of, for example, betaines, N-alkylamidobetaines and their derivatives, glycine derivatives, sultaines, alkyl polyaminocarboxylates, alkylamphoacetates, and their mixtures.

Mention may be made, as betaines, of, for example, cocobetaine, such as the product sold under the name Dehyton AB-30 by Henkel, laurylbetaine, such as the product sold under the name Genagen KB by Clariant, oxyethylenated (10 EO) laurylbetaine, such as the product sold under the name Laurylether(10 EO)betaine by Shin Nihon Rica, or oxyetbylenated (10 EO) stearylbetaine, such as the product sold under the name Stearylether(10 EO)betaine by Shin Nihon Rica.

Mention may be made, among N-alkylamidobetaines and their derivatives, of, for example, cocamidopropyl betaine, sold under the name Lebon 2000 HG by Sanyo or sold under the name Empigen BB by Albright & Wilson, or lauramidopropyl betaine, sold under the name Rewoteric AMB 12P by Witco.

Mention may be made, as sultaines, of cocoylamidopropylhydroxysulphobetaine, sold under the name Crosultaine C-50 by Croda.

Mention may be made, as alkyl polyaminocarboxylates (APAC), of sodium cocoylpolyaminocarboxylate, sold under the name Ampholak 7 CX/C and Ampholak 7 CX by Akzo Nobel, sodium stearylpolyamidocarboxylate, sold under the name Ampholak 7 TX/C by Akzo Nobel, or sodium carboxymethyloleylpolypropylamine, sold under the name Ampholak X07/C by Akzo Nobel.

Mention may be made, as alkylamphoacetates, of, for example, N-disodium N-15cocoyl-N-carboxymethoxyethyl-N-(carboxymethyl)ethylenediamine (CTFA name: disodium cocamphodiacetate), such as the product sold under the name Miranol C2M Concentré NP by Rhodia Chimie, and N-sodium N-cocoyl-N-hydroxyethyl-N-(carboxymethyl)ethy-lene-diamine (CTFA name: sodium cocamphoacetate).

When the composition according to the invention is in a solid particulate form, preferably a pulverant form, it is preferable, among surfactants, to use those which can exist in the powder form, which are, for example, sodium cocoylisethionate, ricinoleic monoethanolamide monosulphosuccinate, sold under the name Rewoderm S 1333 by Witco, monosodium N-lauroyl-L-glutamate (Amisoft LS-11) and sodium palmitoyl methyltaurate, sold under the name Nikkol PMT by Nikkol.

The aqueous phase can optionally, in addition, comprise a gelling agent at a concentration by mass of 0.01 to 20% of the total weight of the composition, provided that the bath is fluid at a temperature of less than 25° C.

In the foaming compositions of the invention, in the liquid fluid form, the aqueous phase is preferably composed of a physiologically acceptable medium which makes possible a topical application and in particular a cosmetic application.

In the present application, the term “physiologically acceptable medium” is understood to mean a medium which is compatible with any keratinous substance, such as the skin, including the scalp, the nails, the mucous membranes, the eyes and the hair or any other keratinous region of the body.

The physiologically acceptable medium of the foaming compositions of the invention preferably comprises water. The amount of water can range from 30 to 99.98% by weight and preferably from 40 to 95% by weight, with respect to the total weight of the composition.

The water used can, in addition to water, be a floral water, such as cornflower water, a mineral water, such as water from Vittel, water from Lucas or water from la Roche Posay, and/or a thermal water.

The physiologically acceptable medium can comprise, in addition to water, one or more solvents chosen from lower alcohols comprising from 1 to 8 carbon atoms, such as ethanol; polyols, such as glycerol; glycols, such as butylene glycol, isoprene glycol, propylene glycol or polyethylene glycols, such as PEG-8; sorbitol; sugars, such as glucose, fructose, maltose, lactose and sucrose; and their mixtures. The amount of solvent(s) can range from 0.5 to 30% by weight and preferably from 5 to 20% by weight, with respect to the total weight of the composition.

The gelling compositions of the invention can also comprise adjuvants known in the cosmetics and dermatological fields, such as inorganic or organic fillers, hydrophilic or lipophilic active principles, preservatives, gelling agents, plasticizers, antioxidants, fragrances, odour absorbers, anticaking agents (agents which prevent the agglomeration of solid particles), sequestering agents (EDTA), acidic or basic pH regulators or buffers, and colouring materials (pigments or dyes or pearlescent agents). The amounts of these various additives are those conventionally used in the fields under consideration, for example from 0.01 to 20% of the total weight of the composition. Of course, a person skilled in the art will take care to choose the optional compound or compounds to be added to the foaming compositions according to the invention so that the advantageous properties intrinsically attached to these compositions are not, or not substantially, detrimentally affected by the envisaged addition.

The term “fillers” should be understood as comprising colourless or white, inorganic or synthetic and lamellar or nonlamellar particles intended to give body or rigidity to the composition and/or softness, mattness and uniformity to make-up. Mention may in particular be made, as fillers, of talc, mica, silica, boron nitride, bismuth oxychloride, kaolin, Nylon powders, such as Nylon-12 powder (Orgasol, sold by Atochem), polyethylene powders, Teflon (tetrafluoroethylene polymer powders), polyurethane powders, polystyrene powders, polyester powders, optionally modified starch, copolymer microspheres, such as those sold under the names Expancel by Nobel Industry, microsponges, such as Polytrap, sold by Dow Corning, silicone resin microbeads, such as those sold by Toshiba under the name Tospearl, precipitated calcium carbonate, magnesium carbonate, basic magnesium carbonate, hydroxyapatite, hollow silica microspheres (Silica Beads from Maprecos), glass or ceramic microcapsules, metal soaps derived from organic carboxylic acids having from 8 to 22 carbon atoms, preferably from 12 to 18 carbon atoms, for example zinc, magnesium or lithium stearate, zinc laurate or magnesium myristate, and their mixtures.

The term “pigments” should be understood as comprising white or coloured and inorganic or organic particles which are insoluble in the medium and which are intended to colour and/or opacify the composition. They can be white or coloured, inorganic and/or organic and of standard or nanometric size. Mention may be made, among inorganic pigments and nanopigments, of titanium dioxide, zirconium dioxide or cerium dioxide, along with zinc oxide, iron oxide or chromium oxide, nanotitaniums (titan dioxide nanopigments), nanozincs (zinc oxide nanopigments) or ferric blue. Mention may be made, among organic pigments, of carbon black and lakes, such as calcium, barium, aluminium or zirconium salts of acid dyes, such as haloacid, azo or anthraquinone dyes.

The term “pearlescent agents” should be understood as comprising iridescent particles which reflect light. Mention may be made, among pearlescent agents which can be envisaged, of natural mother-of-pearl, mica covered with titanium oxide, with iron oxide, with natural pigment or with bismuth oxychloride, and coloured titanium oxide-coated mica.

A gelling agent (other than the gelling compound or polymer already described above, the gelling properties on heating of which are reversible with temperature) can be added to the compositions of the invention, when they are fluid, in order to adjust the texture of the composition and to gain access to a wide range of textures ranging from milk to cream. The gelling compound concerned here is a conventional gelling compound exhibiting gelling properties independent of the temperature. As has already been mentioned above, the compositions according to the invention can, at low temperature, for example at ambient temperature, “in the jar”, assume any desirable texture. There is no restriction on the texture which the composition may have before use.

In particular, the composition does not necessarily have to comprise a gelling agent other than the compound or polymer in order, during use, to obtain a gelled foam. This is because, by virtue of the specific polymer or compound included in the composition of the invention, the foam obtained during the increase in temperature which takes place, for example, when the composition is used, is gelled and stable, whatever the texture or the form of the bath composition before use. A gelling agent will therefore only be included in the composition if it is desired for the latter to have a gelled appearance, this appearance being, according to the invention, only one particular appearance among the multitude of appearances, textures and forms which the bath composition may have.

According to a preferred embodiment of the invention, the composition has no or little gelling agent (less than 0.1%).

The gelling agents which can be used can be hydrophilic gelling agents. Mention may be made, as examples of hydrophilic gelling agents, of, in particular, carboxyvinyl (carbomer) polymers, acrylic copolymers, such as acrylate/alkylacrlyate copolymers, polyacrylamides, such as, for example, the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysor-bate 80) by Seppic; polymers and copolymers of 2-acrylamido-2-methylpropanesulphonic acid, which are optionally crosslinked and/or neutralized, such as the poly(2-acrylamido-2-methylpropane sulphonic acid) sold by Hoechst under the trade name “Hostacerin AMPS”(CTFA name: ammonium polyacryldimethyltauramide); polysaccharides, natural gums, such as xanthan gum, clays and their mixtures.

The composition, when it is fluid, can optionally comprise an oily phase.

This oily phase preferably comprises at least one oil.

Oils which can be used in the composition of the invention include for example:

hydrocarbon-based oils of animal origin, such as perhydrosqualene;

hydrocarbon-based oils of vegetable origin, such as liquid triglycerides of fatty acids comprising from 4 to 10 carbon atoms, such as triglycerides of heptanoic acid or octanoic acid, or, for example, sunflower oil, maize oil, soybean oil, cucumber oil, grape seed oil, sesame oil, hazelnut oil, apricot oil, macadamia oil, arara oil, castor oil, avocado oil, triglycerides of caprylic/capric acids, such as those sold by Stearineries Dubois or those sold under the names Miglyol 810, 812 and 818 by Dynamit Nobel, jojoba oil or karite butter oil;

synthetic esters and ethers, in particular of fatty acids, such as oils of formulae R¹COOR² and R¹OR² in which R¹ represents the residue of a fatty acid comprising from 8 to 29 carbon atoms and R² represents a branched or unbranched hydrocarbon-based chain comprising from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;

linear or branched hydrocarbons of mineral or synthetic origin, such as volatile or nonvolatile liquid paraffins and their derivatives, liquid petrolatum, polydecenes or hydrogenated polyisobutene, such as parleam oil;

natural or synthetic essential oils, such as, for example, eucalyptus oil, lavandin oil, lavender oil, vetiver oil, litsea cubeba oil, lemon oil, sandalwood oil, rosemary oil, camomile oil, savoury oil, nutmeg oil, cinnamon oil, hysope oil, caraway oil, orange oil, geraniol oil, cade oil and bergamot oil;

fatty alcohols having from 8 to 26 carbon atoms, such as cetyl alcohol, stearyl alcohol and their mixture (cetostearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluorinated oils, such as those disclosed in the document JP-A-2-295912;

silicone oils, such as volatile or nonvolatile polymethylsiloxanes (PDMS) comprising a linear or cyclic silicone chain which are liquid or pasty at ambient temperature, in particular cyclopolydimethylsiloxanes (cyclomethicones), such as cyclohexasiloxane; polydimethylsiloxanes comprising pendant alkyl, alkoxy or phenyl groups or alkyl, alkoxy or phenyl groups at the end of the silicone chain, which groups have from 2 to 24 carbon atoms; or phenylated silicones, such as phenyl trimethicones, phenyl dimethicones, phenyltrimethylsiloxydiphenylsiloxanes, diphenyl dimethicones, diphenylmethyldiphenyl-trisiloxanes, 2-phenylethyltrimethylsiloxysilicates and polymethylphenylsiloxanes;

their mixtures.

The term “hydrocarbon-based oil” is understood to mean, in the list of the oils mentioned above, any oil predominantly comprising carbon and hydrocarbon atoms and optionally ester, ether, fluoro, carboxylic acid and/or alcohol groups.

The other fatty substances which can be present in the oily phase are, for example, fatty acids comprising from 8 to 30 carbon atoms, such as stearic acid, lauric acid, palmitic acid and oleic acid; waxes, such as lanolin, beeswax, carnauba wax, candelilla wax, paraffin wax, lignite wax, microcristalline waxes, ceresin, ozokerite or synthetic waxes, such as polyethylene waxes or Fischer-Tropsch waxes; gums, such as silicone gums (dimethiconol); silicone resins, such as trifluoromethyl (C₁₋₄)alkyl dimethicone and trifluoropropyl dimethicone; and silicone elastomers, such as the products sold under the names “KSG” by Shin-Etsu, under the names “Trefil”, “BY29” or “EPSX” by Dow Coming or under the names “Gransil” by Grant Industries.

These fatty substances can be chosen in a manner varied by a person skilled in the art in order to prepare a composition having the desired properties, for example of consistency or of texture.

When it is present, the amount of oily phase can range, for example, from 0.01 to 50% by weight and preferably from 0.1 to 30% by weight, with respect to the total weight of the composition.

The composition according to the invention can constitute a bath gel, bath pearls, a bath powder, a bath milk, a bath cream, and the like.

Starting from the composition according to the invention, that is to say from a composition capable of foaming but which does not foam in particular in the container in which it is held during storage, a foam, that is to say a dispersion of gas bubbles in a continuous aqueous phase, is obtained by exerting a mechanical action, such as stirring, on the said composition or rather on the bath to which the composition according to the invention, whether fluid or solid, has been added.

This mechanical action can be due in particular to the stirring of the bath by the user and/or by a jet or stream of water or by any other means which allows the composition or the volume of the bath to be mixed with a stream of gas to form bubbles of this gas in the aqueous phase. The gas included in the foam is therefore generally air.

The bath compositions according to the invention can, for example, be used in the following way:

the composition according to the invention is introduced into a volume of water intended to form the bath; this mass or volume of water may already be at a “high” temperature greater than the gelling temperature or else at a lower temperature;

the combined volume of water is stirred so as to homogeneously distribute the composition of the invention in the volume of water.

In the case where the temperature of the water is sufficiently high, a foam-gel will then immediately be formed, the foam developing because of the stirring. If the gel is not formed, it is necessary to add an additional amount of composition according to the invention to the mass of water in order to arrive within the concentration ranges where gelling occurs. If the temperature of the water is insufficient, it is possible, by addition of water, for example very hot water, to arrange for the bath temperature to exceed the gelling temperature, in order to give the desired gel texture, care being taken to add the sufficient amount of composition according to the invention to the volume of the bath.

The temperature of the gelled bath will therefore generally be from 30 to 50° C.

The user can be present in the bath prior or subsequent to the introduction of the composition of the invention into the latter.

The temperature of the bath will in fact slowly decline and will change, for example, from a temperature of 30 to 50° C., which is that of the gelled bath, to a temperature of 5 to below 30° C., at which the bath is fluidized and has a greatly reduced viscosity, due to the disappearance of the reversible heat-gelling effect of the polymer or compound included in the bath composition according to the invention. This decrease in temperature occurs over a period of time which generally can correspond to the duration of bathing.

It is, of course, possible to accelerate this cooling and the concomitant fluidizing of the bath by adding relatively colder water to the bath, so as to lower the temperature thereof down to the ranges where the heat-gelling effect no longer exists.

Once the bath has become fluid again, with a low viscosity, it can be easily discharged from the receptacle, such as a bathtub, in which it is found, generally under the effect of gravity alone or with the assistance of rinsing with cold water for example down a drain.

Other characteristics and advantages of the invention will become more clearly apparent on reading the description which follows, given by way of illustration and without implied limitation.

The following examples illustrate the preparation of gelling compositions according to the invention comprising the preferred polymers comprising water-soluble units and specific LCST units.

The polymers used in these examples are composed of a polyacrylic acid (PAA) backbone carrying side chains or grafts composed of specific LCST units. They are characterized by the molar mass of the water-soluble backbone (polyacrylic acid), the chemical nature of the LCST chains, their proportion by mass in the polymer and their molar mass.

The characteristics of the polymers used are given in Table I.

TABLE I

| | | | Proportion: | Degree |
|---|---|---|---|---|
| | | | LCST units in | of |
| | Water-soluble | Grafts | final polymer | grafting |
| | backbone | (LCST units) | (by weight) | (mol %) |
| Polymer 1 | Polyacrylic acid; | (EO)₆(PO)₃₉ | 51% | 3.9% |
| | MW = 450 000 | random | | |
| | | Jeffamine | | |
| | | M-2005; | | |
| | | MW = 2 600 | | |
| Polymer 2 | Polyacrylic acid; | Poly-N- | 49% | 0.9% |
| | MW = 550 000 | isopropyl- | | |
| | | acrylamide | | |
| | | (pNIPAM) | | |
| | | MW = 10 000 | | |
| Polymer 3 | Polyacrylic acid; | (EO)₆(PO)₃₉ | 59% | 5.3% |
| | MW = 450 000 | random | | |
| | | Jeffamine | | |
| | | M-2005; | | |
| | | MW = 2 600 | | |

These polymers are prepared in the following way.

Preparation of Polymer 1

3 g of polyacrylic acid with an average molar mass of 450 000 g/mol (Aldrich) are dissolved in 220 ml of N-methylpyrrolidone in a 500 ml reactor equipped with a reflux condenser, with stirring at 60° C. for 12 h.

4.181 g of monoaminated random (EO)₆(PO)₃₉ copolymer, with a molar mass of 2 600 g/mol and with a cloud point, at a concentration of 1% by weight in water, of 16° C. (Jeffamine M-2005 from Huntsman), are dissolved in 50 ml of N-methylpyrrolidone with stirring at 20° C. for 15 min. The solution obtained is added dropwise to the reaction medium comprising the polyacrylic acid with vigorous stirring at 60° C.

2.158 g of dicyclohexylcarbodiimide are dissolved in 30 ml of N-methylpyrrolidone with stirring at 20° C. for 15 min. The solution obtained is added dropwise to the reaction medium comprising the polyacrylic acid and the monoaminated random (EO)₆(PO)₃₉ copolymer with vigorous stirring at 60° C. The final mixture is stirred for 12 h at 60° C.

The mixture is cooled to 20° C. and then it is placed in a refrigerator at 4° C. for 24 h. The dicyclohexylurea crystals formed are removed by filtering the reaction medium.

The polymer is then neutralized using 19 g of 35% sodium hydroxide solution (fourfold excess with respect to the number of moles of acrylic acid), which leads to its precipitation. After standing for 12 h, the reaction medium is filtered in order to recover the precipitated polymer. The latter is dried under vacuum at 35° C. for 24 h.

13.55 g of solid are recovered and are dissolved in 2 1 of deionized water. This solution is ultrafiltered using a Millipore ultrafiltration system comprising a membrane with a cut-off threshold set at 10 000 daltons. The solution thus purified is lyophilized in order to collect the polymer in the solid form.

7.05 g of poly(sodium acrylate) (450 000 g/mol), grafted with 3.9% (in moles) of monoaminated random (EO)₆(PO)₃₉ copolymer, are obtained.

The proportion by mass of the LCST units in the final polymer is 51%.

The polymer thus obtained exhibits a solubility in water at 20° C. of at least 10 g/l.

Preparation of polymer 2

Polymer 2, which comprises poly-N-isopropylacrylamide (pNIPAM) grafts, is prepared by a 2-stage process:

1) Synthesis of pNIPAM oligomers carrying an aminated reactive end

8 g of N-isopropylacrylamide and 80 ml of dimethyl sulphoxide are introduced into a 250 ml three-necked round-bottomed flask equipped with a reflux condenser and a nitrogen inlet. This mixture is heated with stirring to 29° C. using a water bath and is placed under nitrogen sparging. After 45 min, 0.161 g of aminoethanethiol hydrochloride, dissolved beforehand in 4 ml of dimethyl sulphoxide, are added to the reaction medium. 5 min later, 0.191 g of potassium persulphate, dissolved in 8 ml of dimethyl sulphoxide, is added to the reaction medium. This reaction medium is kept stirred and is maintained under a nitrogen atmosphere for 3 h at 29° C.

The poly-N-isopropylacrylamide (pNIPAM) oligomers synthesized are isolated by precipitation of the reaction medium from a mixture of acetone (40% by volume) and hexane (60%).

2) Grafting of the pNIPAM oligomers to polyacrylic acid

3 g of polyacrylic acid with a molar mass of 550 000 g/mol are dissolved in 100 ml of 1-methyl-2-pyrrolidone in a 250 ml three-necked round-bottomed flask equipped with a reflux condenser, with stirring at 60° C. for 12 h. 3.757 g of pNIPAM oligomers, dissolved beforehand in 25 ml of 1-methyl-2-pyrrolidone, are introduced dropwise into the reaction medium with stirring. 15 min later, 0.776 g of dicyclohexylcarbodiimide, dissolved beforehand in 25 ml of 1-methyl-2-pyrrolidone, are introduced dropwise into the reaction medium with vigorous stirring. The reaction medium is kept stirred for 12 h at 60° C.

The reaction medium is then cooled to 20° C. and then placed in a refrigerator at 4° C. for 24 h. The dicyclohexylurea crystals formed are then removed by filtration. The polymer is then neutralized using 19 g of 35% sodium hydroxide solution (fourfold excess with respect to the number of moles of acrylic acid), which leads to its precipitation. After standing for 12 h, the reaction medium is filtered in order to recover the precipitated polymer. The latter is dried under vacuum at 35° C. for 24 h.

10.2 g of solid are recovered and are dissolved in 2 liters of deionized water. This solution is ultrafiltered using a Millipore ultrafiltration system comprising a membrane with a cut-off threshold set at 10 000 daltons. The solution thus purified is lyophilized in order to collect the polymer in the solid form.

4.8 g of poly(sodium acrylate) (550 000 g/mol), grafted with 0.9% (in moles) of poly-N-isopropylacrylamide, are obtained.

The proportion by mass of the LCST units in the final polymer is 49%.

Polymer 3, in which the grafts, which are the same as in Polymer 1, result from a monoaminated random (EO)₆(PO)₃₉ copolymer (Jeffamine M-2005), was prepared in a way similar to Example 1.

The critical aggregation concentrations (CACs) of the three polymers prepared above are determined by rheology, according to the method described above, using a Haake RS 150 rheometer equipped with cone/plate geometry (35 mm, 2°) and with a thermostatically-controlled bath, in order to control the temperature between 5 and 80° C. The measures were made in the flow mode at a shear rate of 10 s¹, the temperature being varied from 15 to 50° C. at a rate of 0.5° C./minute.

For Polymer 1 in 0.2M NaCl; the critical aggregation concentration (CAC) is approximately 1% by weight.

For Polymer 2 in pure water, the critical aggregation concentration is approximately 0.3% by weight.

While for Polymer 3 in 0.2M NaCl, the critical aggregation concentration (CAC) is approximately 1% by weight.

The Theological measurements made in the following examples were carried out in the same way and under the same conditions as those carried out to determine the CAC values but while varying the temperature within the range from 20 to 35° C., at a rate of 0.5° C./min.

### EXAMPLE 1

a) This example describes a bath composition comprising 5% by weight of Polymer 1.

| | |
|---|---|
| Polymer 1 | 5 g |
| NaCl | 1.17 g |
| Demineralized water | 93.83 g |

This composition is prepared by simple introduction of the polymer into salt water with stirring for 2 h.

b) Gelling Properties and Removal from the Bath on Cooling

The gelling properties of the composition prepared above and its fluidization on cooling were demonstrated by rheological measurements.

The bath composition obtained according to the invention exhibits the following viscosities:

Viscosity at 20° C.(10 s⁻¹)=0.015 Pa.s.

Viscosity at 35° C.(10 s⁻¹)=10 Pa.s

Viscosity at 20° C., after heating to 35° C. (10s⁻¹): 0.015 Pa.s.

The gelling power of Polymer 1 makes it possible to obtain a gelled bath at 35° C.

When the temperature decreases from 35° C. to 20° C., the composition fluidizes and can thus be easily removed.

### EXAMPLE 2

This example describes a bath composition comprising 1% by weight of Polymer 2.

a) Composition

| | |
|---|---|
| Polymer 2 | 1 g |
| Demineralized water | 99 g |

This composition is prepared by simple introduction of the polymer into water with stirring for2h.

b) Gelling Properties and Removal from the Bath on Cooling

The gelling properties of the composition prepared above and its fluidization on cooling were demonstrated by Theological measurements.

The bath composition obtained according to the invention exhibits the following viscosities:

Viscosity at 20° C.(10 s⁻¹)=0.17 Pa.s.

Viscosity at 35° C.(10 s⁻¹)=0.7 Pa.s

Viscosity at 20° C., after heating to 35° C.(10 s⁻¹): 0.16 Pa.s.

The gelling power of Polymer 2 makes it possible to obtain a gelled bath at 35° C.

When the temperature decreases from 35° C. to 20° C., the composition fluidizes and can thus be easily removed.

### EXAMPLE 3

This example describes a bath composition comprising 5% by weight of Polymer 3 and 3% of an alkylpolyglycoside surfactant.

The surfactant used is a (C₁₀₋₁₄ alkyl)polyglycoside, sold under the name Oramix® NS10 by Seppic®.

a) Composition

| | |
|---|---|
| Polymer 3 | 5 g |
| Oramix ® N510 | 3 g |
| Demineralized water | 92 g |

This composition is prepared by mixing equal proportions of a 10% solution of Polymer 3 and of a 6% solution of Oramix® NS10. These two mother solutions are prepared by simple introduction of the polymer (or of the surfactant) into water with stirring for 2 h.

b) Gelling Properties and Removal from the Bath on Cooling

The gelling properties of the composition prepared above and its fluidization on cooling were demonstrated by rheological measurements.

The composition of the invention exhibits the following viscosities:

Viscosity at 20° C.(10 s⁻¹)=0.06 Pa.s.

Viscosity at 35° C.(10 s⁻¹)=0.8 Pa.s

Viscosity at 20° C., after heating to 35° C. (10 s⁻¹): 0.07 Pa.s.

The gelling power of Polymer 3 makes it possible to obtain a gelled bath at 35° C.

When the temperature decreases from 35° C. to 20° C., the composition fluidizes and can thus be easily removed.

### EXAMPLE 4

This example describes a heat-gelling system and a bath composition comprising an associative polymer in combination with a surfactant which forms bilayer structures in aqueous solution.

a) Heat-gelling system

Poly(sodium acrylate) with a molar mass of 150 000 g/mol, grafted with 3% (in moles) of dodecyl chains, combined with the ethoxylated alkyl surfactant C₁₂(EO)₄.

b) Bath Composition

| | |
|---|---|
| Polymer | 1 g |
| C₁₂(EO)₄ | 3.24 g |
| Demineralized water | 95.76 g |

This composition is prepared by simple mixing of a 2% solution of the polymer and of a 6.48% solution of the surfactant. These two mother solutions are prepared by simple introduction of the polymer (or of the surfactant) into water with stirring.

c) Gelling Properties and Removal from the Bath on Cooling

The gelling properties of the composition prepared above and its fluidization on cooling were demonstrated by rheological measurements.

The composition according to the invention exhibits the following viscosities:

| | |
|---|---|
| Viscosity at 20° C. (10 s⁻¹) | 1 Pa.s |
| Viscosity at 35° C. (10 s⁻¹) | 40 Pa.s |
| Viscosity at 20° C., after heating | 1 Pa.s |
| to 35° C. (10 s⁻¹) | |

The gelling power of the polymer +surfactant system makes it possible to obtain a gelled bath at 35° C. When the temperature decreases from 35° C. to 20° C., the composition fluidizes and can easily be removed.

### EXAMPLE 5

This example describes a gelling system and a bath composition comprising methylcellulose.

a) Heat-gelling System

Methocel® A100, supplied by Dow Chemical; its degree of substitution by methoxy groups is between 1.6 and 1.8.

b) Bath Composition

| | |
|---|---|
| Polymer | 5 g |
| Demineralized water | 95 g |

This composition is prepared by simple introduction of the polymer into water with stirring.

c) Gelling Properties and Removal from the Bath on Cooling

The gelling properties of the composition prepared above and its fluidization on cooling were demonstrated by rheological measurements.

The composition of the invention exhibits the following viscosities:

| | |
|---|---|
| Viscosity at 20° C. (10 s⁻¹) | 1 Pa.s |
| Viscosity at 40° C. (10 s⁻¹) | 10 Pa.s |
| Viscosity at 20° C., after heating | 0.9 Pa.s |
| to 40° C. (10 s⁻¹) | |

The gelling power of this polymer makes it possible to obtain a gelled bath at 40° C. When the temperature decreases from 40° C. to 200° C., the composition fluidizes and can easily be removed.

### EXAMPLE 6

This example describes a heat-gelling system and a bath composition comprising a PEO/PPO/PEO block copolymer.

a) Heat-gelling System

Pluronic P-85 supplied by BASF; its molar mass is 4 500 g/mol; the molar mass of the PEO blocks is equal to 1 125 g/mol and that of the PPO block is equal to 2 250 g/mol.

b) Bath Composition

| | |
|---|---|
| Polymer | 27 g |
| Demineralized water | 73 g |

This composition is prepared by simple introduction of the polymer into water with stirring.

c) Gelling Properties and Removal from the Bath on Cooling

The gelling properties of the composition prepared above and its fluidization on cooling were demonstrated by rheological measurements.

The composition of the invention exhibits the following viscosities:

| | |
|---|---|
| Viscosity at 20° C. (10 s⁻¹) | 0.03 Pa.s |
| Viscosity at 35° C. (10 s⁻¹) | 150 Pa.s |
| Viscosity at 20° C., after heating | 0.04 Pa.s |
| to 35° C. (10 s⁻¹) | |

The gelling power of this polymer makes it possible to obtain a gelled bath at 35° C. When the temperature decreases from 35° C. to 20° C., the composition fluidizes and can easily be removed.

### REFERENCES

[1] N. Sarkar, J. Appl. Polym. Sci., 1979, 24,1 073.

[2] W. Brown, K. Schillen, M. Almgren, S. Hvidt, P. Bahadur, J. Phys. Chem., 1991, 95, 1 850.

[3] A. Carlsson, G. Karlstrom, B. Lindman, Coll. Surf., 1990, 47, 147.

[4] K. Loyen, I. Iliopoulos, R. Audebert, Langmuir, 1994, 10, 1 421.

[5] D. Hourdet et al., Polymer, 1994, Vol. 35, No. 12, pp. 2 624-2 630.

[6] F. L'Alloret et al., Coll. Polym. Sci., 1995, Vol. 273, pp. 1 1⁶3-1 173.

[7] F. L'Alloret, Revue de l'Institut Français du Pétrole, 1997, Vol. 52, No. 2, pp. 117-128.

[8] EP-A-0 583 814.

[9] EP-A-0 629 649.

[10] WO-A-95 24430.

[11] U.S. Pat. No. 5,939,485.

[12] WO-A-97 00275.

[13] WO-A-98 48768.

[14] WO-A-99 35961.

[15] Articles by Taylor et al., Journal of Polymer Science, Part A: Polymer Chemistry, 1975, 13, 2 551.

[16] J. Bailey et al., Journal of Applied Polymer Science, 1959, 1, 56.

[17] Heskins et al., Journal of Macromolecular Science, Chemistry A2, 1968, 1 441.

[18] EP-A-0 566 438.

[19] FR-A-2 739 556.

French patent application 01 05 112 filed Apr. 13, 2001 is incorporated herein by reference, as are all documents, references, standards, texts, patents, publications and articles mentioned above.

1. A gelling bath composition comprising at least one heat-gelling polymer or compound having gelling properties on heating that are reversible with temperature.

2. The composition according to claim 1, in which said heat-gelling polymer or compound is selected from the group consisting of the following compounds or polymers:
water-soluble polymers comprising water-soluble units and units exhibiting, in water, a lower critical solution temperature (LCST);
methylcellulose and hydroxypropylmethylcellulose;
copolymers with polyoxyalkylenated blocks;
nonionic cellulose ethers;
associative water-soluble polymers in the presence of at least one surface-active agent which forms bilayer structures in aqueous solution.

3. The composition according to claim 2, in which said heat-gelling polymer or compound is a water-soluble polymer comprising water-soluble units and units exhibiting, in water, a lower critical solution temperature LCST.

4. The composition according to claim 3, in which a demixing temperature on heating in aqueous solution of said LCST units of the polymer is from 5 to 50° C. for a concentration by mass in water of said units of 1%.

5. The composition according to claim 3, in which a demixing temperature on heating in aqueous solution of the LCST units of the polymer is from 15 to 40° C. for a concentration by mass in water of said units of 1%.

6. The composition according to claim 3, in which the polymer exists in the form of a block polymer comprising blocks comprising water-soluble units alternating with blocks composed of LCST units, or in the form of a graft polymer, the backbone of which is formed from water-soluble units, the said backbone carrying grafts composed of LCST units, said polymers optionally partially crosslinked.

7. The composition according to claim 3, in which the water-soluble units are capable, in all or in part, of being obtained by polymerization or by polycondensation or are composed, in all or in part, of natural or modified natural polymers.

8. The composition according to claim 7, in which the water-soluble units are capable, in all or in part, of being obtained by polymerization of at least one monomer selected from the group of following monomers:
1) (meth)acrylic acid;
2) vinyl monomers of following formula (I): 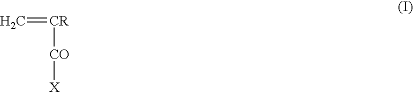
in which:
R is chosen from H, —CH₃, —C₂H₅ or —C₃H₇; and
X is chosen from:
alkyl oxides of —OR′ type, where R′ is a saturated or unsaturated and linear or branched hydrocarbon-based radical having from 1 to 6 carbon atoms, optionally substituted by at least one halogen atom (iodine, bromine, chlorine, fluorine); or one group of the following types: sulphonic (—SO₃⁻), sulphate (—SO₄⁻) or phosphate (—PO₄H₂); hydroxyl (—OH); primary amine (—NH₂); or secondary (—NHR₁), tertiary (—NR₁R₂) or quaternary (—N⁺R₁R₂R₃) amine with R₁, R₂ and R₃ being, independently of one another, a saturated or unsaturated and linear or branched hydrocarbon-based radical having 1 to 6 carbon atoms, with the proviso that the sum of the carbon atoms of R′+R₁+R₂+R₃ does not exceed 7; and
—NH₂, —NHR₄ and —NR₄R₅ groups in which R₄ and R₅ are, independently of one another, saturated or unsaturated and linear or branched hydrocarbon-based radicals having 1 to 6 carbon atoms, with the proviso that the total number of carbon atoms of R₄+R₅ does not exceed 7, the said R₄ and R₅ radicals optionally being substituted by a halogen atom (iodine, bromine, chlorine, fluorine); or a group of the following types: hydroxyl (—OH); sulphonic (—SO₃⁻); sulphate (—SO₄⁻); phosphate (—PO₄H₂); primary amine (—NH₂); or secondary (—NHR₁), tertiary (—NR₁R₂) and/or quaternary (—N⁺R₁R₂R₃) amine with R₁, R₂ and R₃ being, independently of one another, a saturated or unsaturated and linear or branched hydrocarbon-based radical having 1 to 6 carbon atoms, with the proviso that the sum of the carbon atoms of R₄+R₅+R₁+R₂+R₃ does not exceed 7;
3) maleic anhydride;
4) itaconic acid;
5) vinyl alcohol of formula CH₂═CHOH;
6) vinyl acetate of formula CH₂═CH-OCOCH₃;
7) N-vinyllactams;
8) vinyl ethers of formula CH₂=CHOR₆, in which R₆, is a saturated or unsaturated and linear or branched hydrocarbon-based radical having from 1 to 6 carbon atoms;
9) water-soluble styrene derivatives;
10) dimethyldiallylammonium chloride; and
11) vinylacetamide.

9. The composition according to claim 8, in which the monomer of formula (I) is chosen from 2-acrylamido-2-methylpropanesulphonic acid (AMPS) and (meth)acrylic acid.

10. The composition according to claim 7, in which the water-soluble units of the polymer are composed, in all or in part, of polycondensates or of natural or modified natural polymers chosen from one or more of the following components:
water-soluble polyurethanes;
xanthan gum;
alginates and their derivatives;
cellulose derivatives;
galactomannans and their derivatives; and
polyethyleneimine.

11. The composition according to claim 7, in which the water-soluble units of the polymer have a molar mass ranging from 1 000 g/mol to 50 000 000 g/mol, when they constitute the water-soluble backbone of a graft polymer, or a monomer mass ranging from 500 g/mol to 100 000 g/mol, when they constitute a block of a multiblock polymer.

12. The composition according to claim 3, in which the LCST units of the polymer are comprised of one or more polymers selected from the following group of polymers:
polyethers;
poly(vinyl methyl ether)s;
N-substituted polymeric and copolymeric acrylamide derivatives having an LCST; and
poly-N-vinylcaprolactam and copolymers of N-vinylcaprolactam.

13. The composition according to claim 3, in which the LCST units of the polymer are comprised of poly(propylene oxide) (PO)ₙ, where n is an integer from 10 to 50, or of random copolymers of ethylene oxide (EO) and of propylene oxide (PO), represented by the formula:
(EO)ₘ(PO)ₙ
in which m is an integer ranging from 1 to 40, preferably from 2 to 20, and n is an integer ranging from 10 to 60, preferably from 20 to 50.

14. The composition according to claim 13, in which the molar mass of the LCST units of the polymer is from 500 to 5 300 g/mol.

15. The composition according to claim 3, in which the LCST units of the polymer are comprised of a polymer chosen from poly-N-isopropylacrylamide, poly-N-ethylacrylamide and copolymers of N-isopropylacrylamide or of N-ethylacrylamide and of a vinyl monomer chosen from maleic anhydride, itaconic acid, vinylpyrrolidone, styrene and its derivatives, dimethyldiallylammonium chloride, vinylacetamide, vinyl alcohol, vinyl acetate, vinyl ethers, vinyl acetate derivatives, and vinyl monomers of following formula (I): 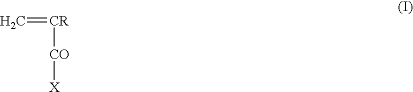
in which:
R is chosen from H, —CH₃, —C₂H₅ or —C₃H₇; and
X is chosen from:
alkyl oxides of —OR′ type, where R′ is a saturated or unsaturated and linear or branched hydrocarbon-based radical having from 1 to 6 carbon atoms, optionally substituted by at least one halogen atom (iodine, bromine, chlorine, fluorine); or one group of the following types: sulphonic (—SO₃⁻), sulphate (—SO₄⁻) or phosphate (—PO₄H₂); hydroxyl (—OH); primary amine (—NH₂); or secondary (—NHR₁), tertiary (—NR₁R₂) or quaternary (—N⁺R₁R₂R₃) amine with R₁, R₂ and R₃ being, independently of one another, a saturated or unsaturated and linear or branched hydrocarbon-based radical having 1 to 6 carbon atoms, with the proviso that the sum of the carbon atoms of R′+R₁+R₂+R₃ does not exceed 7.

16. The composition according to claim 15, in which the molar mass of the LST units of the polymer is from 1 000 g/mol to 500 000 g/mol.

17. The composition according to claim 3, in which the LCST units of the polymer are comprised of a poly-N-vinylcaprolactam or a copolymer of N-vinylcaprolactam and of a vinyl monomer chosen from maleic anhydride, itaconic acid, vinylpyrrolidone, styrene and its derivatives, dimethyldiallylammonium chloride, vinylacetamide, vinyl alcohol, vinyl acetate, vinyl ethers, vinyl acetate derivatives, and vinyl monomers of following formula (I): 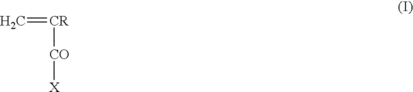
in which:
R is chosen from H, —CH₃, —C₂H₅ or —C₃H₇; and
X is chosen from:
alkyl oxides of —OR′ type, where R′ is a saturated or unsaturated and linear or branched hydrocarbon-based radical having from 1 to 6 carbon atoms, optionally substituted by at least one halogen atom (iodine, bromine, chlorine, fluorine); or one group of the following types: sulphonic (—SO₃⁻), sulphate (—SO₄⁻) or phosphate (—PO₄H₂); hydroxyl (—OH); primary amine (—NH₂); or secondary (—NHR₁), tertiary (—NR₁R₂) or quaternary (—N⁺R₁R₂R₃) amine with R₁, R₂ and R₃ being, independently of one another, a saturated or unsaturated and linear or branched hydrocarbon-based radical having 1 to 6 carbon atoms, with the proviso that the sum of the carbon atoms of R′+R₁+R₂+R₃ does not exceed 7.

18. The composition according to claim 17, in which the molar mass of the LCST units is from 1 000 to 500 000 g/mol.

19. The composition according to claim 3, in which the proportion by mass of the LCST units in the polymer is from 5 to 70% with respect to the polymer.

20. The composition according to claim 1, wherein the composition is in the solid form.

21. The composition according to claim 20, wherein the composition is in the form of particles.

22. The composition according to claim 20, wherein the concentration by weight of the heat-gelling polymer or compound is from 0.5 to 1 g per gram of solid composition.

23. The composition according to claim 1, wherein the composition further comprises a continuous aqueous phase.

24. The composition according to claim 23, in which the continuous aqueous phase exists in the form of a concentrated aqueous solution of polymer or compound.

25. The composition according to claim 23, wherein the concentration by weight of polymer or compound in the aqueous phase is from 0.1 to 90%.

26. The composition according to claim 1, wherein said composition is a foaming composition.

27. The composition according to claim 1, wherein said composition further comprises at least one foaming surface-active agent.

28. The composition according to claim 27, wherein said foaming surface-active agent is nonionic.

29. The composition according to claim 1, wherein said polymer or compound is present in an amount sufficient to gel said bathing water at a predetermined elevated temperature and allow return to a fluid state when the water is cooled.

30. A method comprising mixing bathing water with the composition of claim 1 in a bathing receptacle.

31. A gelled bath comprising gelled water in a bathing receptacle, said water comprising at least one heat-gelling polymer or compound having gelling properties on heating that are reversible with temperature.

32. A method of bathing, comprising bathing in the gelled bath of claim 32.
